(19) United States
(12) Patent Application Publication
(10) Pub. No.: US 2010/0197011 A1
(43) Pub. Date: Aug. 5, 2010

(54) METHOD OF OBTAINING CELL LINES IN A PROTEIN-FREE MEDIUM AND CELL LINES THUS OBTAINED

(75) Inventors: Rolando PEREZ RODRIGUEZ, Ciudad Habana (CU); Adolfo CASTILLO VITLLOCH, Ciudad Habana (CU); Svieta VITORES SARAZOLA, Ciudad Habana (CU); Tammy BOGGIANO AYO, Ciudad Habana (CU); Luis ROJAS DEL CALVO, Ciudad Habana (CU)

Correspondence Address: WALKER & JOCKE, L.P.A., 231 SOUTH BROADWAY STREET, MEDINA, OH, 44256, US

(73) Assignee: CENTRO DE INMUNOLOGIA MOLECULAR, Ciudad de la Habana (CU)

(21) Appl. No.: 12/759,208

(22) Filed: Apr. 13, 2010

Related U.S. Application Data
(62) Division of application No. 10/532,269, filed on Apr. 22, 2005.

(30) Foreign Application Priority Data
Oct. 23, 2002 (CU) 239/2002

(51) Int. Cl. C12N 5/09; C12N 5/071

(52) U.S. Cl. 435/354; 435/325

(57) ABSTRACT

A method of recovering mammalian cell clones adapted to serum and protein-free media is disclosed. The procedure includes a two-stage adaptation process to grow in that condition. A critical protein concentration interval is disclosed in which cells must grow in order to gain the capacity to survive in serum and protein-free condition, once the cells have grown at the critical interval concentrations, subsequent decreases of the concentration will affect neither viability nor cellular doubling time. The critical protein concentration interval is cell line specific. Furthermore, mammalian cells clones are disclosed, which are stable in serum- and protein-free media for at least 40 generations; additionally, clones disclosed express a recombinant product. The cell clones disclosed produce the humanized anti-EGF-R antibody hR3, the humanized anti-CD6 antibody T1hT, the chimeric anti CD3 antibody T3Q, or fragments thereof.

### FIELD OF THE INVENTION

The present invention relates to biotechnology, specifically to a method of recovering stable cell clones adapted to serum- and protein-free medium by a two-stage adaptation process.

### BACKGROUND OF THE INVENTION

Since the development of the in vitro cultivation of mammalian cells the demand for large scale production of these cells has increased due to diagnostic and therapeutic potential of many of the products they produce. These useful agents include monoclonal antibodies, human growth hormone, lymphokines, erythropoietin, blood clotting factors and tissue plasminogen activators.

The use of recombinant monoclonal antibodies (rMab) for therapy and in vivo diagnosis of different diseases imply in many cases the use of high dose treatments. This fact makes necessary the production of large amount of the rMab of interest with a very high purity.

Several recombinant monoclonal antibodies with potential use in cancer and autoimmune diseases therapy and diagnostic have been expressed in NSO myeloma cells at Center of Molecular Immunology. U.S. Pat. No. 5,891,996, describe the obtainment of the chimeric and humanized antibodies against Epidermal Growth Factor receptor (EGF-R), useful in diagnosis and therapy of tumors expressing said receptor. WO 97/19111 describes anti-CD6 monoclonal antibodies useful in diagnosis and therapy in patients suffering psoriasis. Gavilondo et al. in Hybridoma 9 No. 5, 1999 reported an anti-CD3 monoclonal antibody called IOR-T3a

For protein-free culturing conditions, various techniques have been developed. Thus, specifically defined, complete protein-free media have been developed which allow the cell growth under protein-free conditions.

WO 97/05240 describes the expression of recombinant proteins under protein-free conditions.

JP 2696001 describes the use of a protein-free medium for the production of factor VIII in CHO cells by adding a non-ionic surface-active agent or cyclodextrin to increase the productivity of the host cells. To increase the effectiveness of these additives, the addition of, e.g., butyrate and lithium is recommended.

WO 96/26266 describes the culturing of cells in a medium which contains a glutamin-containing protein hydrolysate whose content of free amino acids is less than 15% of the total weight of the protein, and whose peptides have a molecular weight of less than 44 kD. As the culturing medium for the cell cultures, a synthetic minimum medium is used as the basic medium to which, inter alia, fetal calf serum, gentamycine and mercapto-ethanol are added in addition to protein hydrolysate. The use of this serum-containing medium for the recombinant production of blood factors has not been mentioned.

U.S. Pat. No. 5,393,668 A describes special synthetic surfaces which allow the growth of adherent cells under protein-free conditions.

To stimulate cell proliferation, CHO cells which over express human insulin have been multiplied on an artificial substrate to which insulin is covalently bound (Ito et al. 1996 PNAS U.S.A. 93:3598-3601).

Reiter et al. (1992. Cytotechnology 9:247-253) describe the immobilisation of r-CHO cells first grown in serum-containing medium at a high density on carriers, and subsequent perfusion of the immobilized cells in protein-free medium during the production phase, wherein a continuous liberation of protein into the cell culture supernatant was found. There, the cells were maintained for less than 10 generations in protein-free medium.

Previous methods for the successful preparation of a large-scale cell culture under protein-free conditions have been described for continuous cell lines; in particular VERO cells (WO 96/15231). There, the cells are grown under serum- and protein-free conditions from the original ampoule up to a large technical scale of 1200 liters.

To adapt cells initially grown under serum-containing conditions to protein free medium is a rather troublesome process which usually takes long time; in addition, it has repeatedly been found that the yield of expressed protein and the productivity of recombinant CHO cells greatly drops after adaptation in protein-free medium as compared to serum-containing conditions (Paterson et al. 1994. Appl. Microbiol. Biotechnol. 40:691-658). This is the consequence of an instability or reduced growth of the recombinant clones due to the changed culturing conditions. Despite the use of a stable original clone, on account of the altered fermentation conditions, repeatedly a large portion of the cells become cells with reduced expression or also non-producers, which overgrow product producers during the production process, whereby the culture of the fermenter finally largely consists of non-producers or of such cells having a low expression.

In the present invention we have established an approach to develop stable cell lines adapted to serum- and protein-free media. Following this approach several clones were isolated in protein-free medium.

### DETAILED DESCRIPTION OF THE INVENTION

### Two Stage Adaptation of Cell Lines to Protein Free Medium

This procedure comprises mammalian cell lines, for which it's not possible to carry out a direct procedure of adaptation from serum-supplemented or serum-free medium to protein-free medium.

The method of the present invention consists of a two stages process during adaptation of cell lines to protein-free medium (PFM).

The first step which is consider as a Non critical stage: the reduction of protein contents occurs without the lost of cell viability and there is not an important decrease of population doubling time in each step of protein concentration. The non-critical stage is observed usually between 5 and 0.5 mg/mL of total protein concentration in the culture medium and the culture shows almost the same growth rate that in the initial culture medium.

This first stage starts with cell line viability between 80 and 100% and cells are grown in culture media with consecutive protein concentration reduction up to a critical protein concentration at which cell viability drop to 0%. This protein concentration is the start point for the next stage.

The second step which is consider as a Critical stage: At this stage it occurs a decrease in cell viability and population-doubling time of the cells and it will take more time to adapt from one step of protein concentration to another. There exist critical protein concentrations in the culture medium, which is not possible to bypass during the adaptation process. These critical protein concentrations are specific for each recombinant cell line, but usually are below 0.6 mg/mL. Once the cells have recovered the initial viability and growth rate at these critical protein concentrations there is possible to subculture to the follow condition with lower protein concentration.

Once the critical protein concentration is fixed, its closed higher protein concentration which support cells growth is consider the pre-critical protein concentration. Starting from the pre-critical protein concentration it is reduced slowly up to cells recover the initial viability and growth rate.

The selected combination of steps to reduce the protein concentration at the critical stage will determine the total adaptation time in this stage and the rate of adaptation (V_{adapt}.), calculated as the relationship:

$${V}_{adapt .} = \frac{ΔProtein concentration}{Δ {T}_{adapt .}}$$

However this step combination will not have influence upon the time needed for adaptation to each protein concentration, including critical concentrations. In order to determine the end of non-critical stage and the critical protein concentrations it's necessary to carry out an stepwise adaptation, by serial dilutions of the cell culture in the desired protein free medium reducing two fold the protein concentration each time (Table 1). This reduction can be done by the decrease of serum concentration or supplementing the basal medium with different levels of some rich in proteins serum substitute.

TABLE 1
Stepwise reduction of protein concentration in order to determine
critical concentrations starting from a culture medium
supplemented with 5 mg/mL of protein (equivalent to 10% of
fetal bovine serum).

| | Total protein | Equivalent FBS |
|---|---|---|
| Step | concentration | concentration*, |
| Number | mg/mL | % v/v |
| 1 | 5.000 | 10.00 |
| 2 | 2.500 | 5.00 |
| 3 | 1.250 | 2.50 |
| 4 | 0.625 | 1.25 |
| 5 | 0.312 | 0.60 |
| 6 | 0.156 | 0.30 |
| 7 | 0.000 | 0.00 |

Legend:
FBS—Fetal Bovine Serum
PFM—Protein Free Medium
SCM—Serum Containing Medium
*The total protein content of the fetal bovine serum is considered about 50 mg/mL.

Before to start the adaptation procedure the cells should be maintained with more than 80% of viability in T-flasks in the standard medium usually employed to culture the cells.

The adaptation process is carry out step by step following stages described below.
i. Seed 3 wells in the six-well culture plate with recombinant cell line using the standard cell culture medium (with the initial protein concentration). The cell density should be in the range of 1 to 5×10⁵ cells/mL. After 48 hours a half of the supernatant is replaced by fresh protein-free medium, thus rendering a final protein concentration which is 50% of the starting condition.
ii. Each 48 hours the supernatant is completely replaced by fresh culture medium with a protein concentration which is 50% of the starting condition.
iii. The cells are grown to confluence under this protein concentration.
iv. Cells from step iii are seeded in at least 3 wells at a density in the range 1 to 5×10⁵ cells/mL in culture medium with a protein concentration which is 50% of the starting condition. After 48 hours a half of the supernatant is replaced by fresh protein-free medium, rendering a final protein concentration which is 50% of the former condition.
v. Each 48 hours the supernatant is completely replaced by fresh culture medium with a protein concentration which is 50% of the former condition.
vi. The cells are grown to confluence under this protein concentration.
vii. Steps from (iv) to (vi) are repeated, during each cycle the protein concentration is reduced to 50% of the concentration of the previous cycle. This procedure is repeated up to reach a protein concentration which causes cell death.
viii. Cells are seeded from a cell culture with a viability of 80% or higher growing in the pre-critical protein concentration in at least 3 wells at a density in a range of 2 to 6×10⁵ cells/mL. Cells are grown in the pre-critical protein concentration and after 48 hours the 25% of the supernatant is replaced by fresh protein-free medium, thus rendered it a final protein concentration which is the 75% of the pre-critical protein concentration.
ix. Each 48 hours the supernatant is completely replaced by fresh culture medium with a protein concentration which is 75% of the pre-critical protein concentration.
x. The cells are grown to confluence under this protein concentration.
xi. Cells from step (x) are seeded in at least 3 wells at a density in the range 2 to 6×10⁵ cells/mL in culture medium with a protein concentration which is 75% of the pre-critical protein concentration. After 48 hours the 25% of the supernatant is replaced by fresh protein-free medium, thus rendered it a final protein concentration which is 75% of the concentration of the previous step.
xii. Each 48 hours the supernatant is completely replaced by fresh culture medium with a protein concentration which is 75% of the concentration of the concentration in step (x).
xiii. The cells are grown to confluence under this protein concentration.
xiv. Steps from (xi) to (xiii) are repeated, during each cycle the protein concentration is reduced to 75% of the concentration of the previous cycle, then this procedure is repeated up to reach a protein concentration which does not cause any lost of cell viability and decrease in population doubling time. When the cells are transferred to a medium with lower protein concentration and they are able to growth without any lost of cell viability and decrease in population doubling time before the first subculture, we could consider that cells have reached again the non-critical stage and seed it directly in protein-free medium (0 mg/mL of protein concentration).

In the procedure of the present invention the initial culture medium contains a range between 5 to 10% of fetal bovine serum.

The mammalian cell line adapted to growth in protein-free medium is a myeloma, particularly NSO cells.

The present invention also could be useful when using NSO cell line transfected with a polypeptide or a recombinant protein, particularly when they are transfected with a sequence encoding a recombinant antibody or fragments thereof. The mammalian cell lines modified by procedure of the present invention growing in protein-free medium are also disclosed.

In a different embodiment of the present invention it is disclosed any mammalian cell line expressing a humanized or chimeric antibody selected from the group consisting of the anti-EGF receptor hR3, anti-CD6 T1hT, anti-CD3 T3Q antibodies or fragments thereof growing in protein-free medium, thus the antibodies secreted by this cell lines.

The cell lines obtained by the method of the present invention growth in protein-free medium stably for at least 40 generations.

### EXAMPLES

### Example 1

### Adaptation of Recombinant Cell Line hR3 to Protein-Free Medium

The recombinant cell line hR3 was obtained by transfection of the myeloma NSO cell line with the vector constructions to express the light and heavy chains of the humanized anti-EGF human receptor hR3 monoclonal antibody. The adaptation of this cell line to protein-free medium was carried out following the procedure Previously described, by two stage reduction of protein content of the medium.

These cells were cultured in RPMI-1640 medium supplemented with 10% of FBS. The FBS was replaced by adding the protein reach supplement, Nutridoma NS (Boheringer Manheinn) to RPMI-1640 protein-free medium when the protein concentration was 0.15 mg/mL. The reduction of the protein content in the initial medium was done by successive dilutions with PFHM-II protein-free medium (Gibco).

FIGS. 1 and 2 respectively show the results of calculating the critical concentrations and adaptation rates V_{adapt}.

### Example 2

### Adaptation of Recombinant Cell Line T1hT to Protein-Free Medium

The recombinant cell line T1hT was obtained by transfection of the myeloma NSO cell line with the vector constructions to express the light and heavy chains of a humanized by epitope T suppression method anti-human CD6 monoclonal antibody.

The adaptation of this cell line to protein/free medium was carried out following the procedure described in the point 2, by two stage reduction of protein content of the medium.

These cells were initially cultured in RPMI-1640 medium supplemented with 10% of FBS. The reduction in the protein content was done by successive dilution of the initial medium with PFHM-II protein-free medium from Gibco.

The results of calculation of the critical concentrations and adaptation rates V_{adapt}. are showed in the FIGS. 3 and 4 respectively.

### Example 3

### Adaptation of Recombinant Cell Line T3Q to Protein-Free Medium

The recombinant cell line T3Q was obtained by transfection of the myeloma NSO cell line with the vector constructions to express the light and heavy chains of the humanized monoclonal antibody T3Q which recognize the CD3 receptor on human lymphocytes.

The adaptation of this cell line to protein/free medium was carried out following the procedure described in the point 2, by two stage reduction of protein content of the medium.

These cells were initially cultured in RPMI-1640 medium supplemented with 10% of FBS. The reduction in the protein content was done by successive dilution in the PFHM-II protein-free medium from Gibco.

The results of calculation of the critical concentrations and adaptation rates V_{adapt}. are showed in the FIGS. 5 and 6 respectively.

### BRIEF DESCRIPTION OF THE FIGURES

FIG. 1: Correlation of the time needed to adapt the hR3 cells to each protein concentration (up to recover of the viability and doubling time) with the natural logarithm of the inverse of protein concentration. Values of critical concentrations for h-R3 cell line: —0.32 and 0.11 mg/mL of total protein concentration.

FIG. 2: Correlation of the total time from the start of adaptation procedure with the natural logarithm of the inverse of protein concentration for the h-R3 cell line. Values of adaptation rates for h-R3 cell line during critical stage —0.0053 mg/d.

FIG. 3: Correlation of the time needed to adapt the T1hT cells to each protein concentration (up to recovered the viability and doubling time) with the natural logarithm of the inverse of protein concentration. Values of critical concentrations for T1hT cell line: —0.12 and 0.01 mg/mL of total protein concentration.

FIG. 4: Correlation of the total time from the start of adaptation procedure with the natural logarithm of the inverse of protein concentration for the T1hT cell line. Values of adaptation rates for T1hT cell line —0.0014 mg/d.

FIG. 5: Correlation of the time needed to adapt the T3Q cells to each protein concentration (after recovering of the viability and doubling time) with the natural logarithm of the inverse of protein concentration. Values of critical concentrations for T3Q cell line: —0.63 mg/mL of total protein concentration.

FIG. 6: Correlation of the total time from the start of adaptation procedure with the natural logarithm of the inverse of protein concentration for the T3Q cell line. Values of adaptation rates for T3Q cell line —0.0172 mg/d.

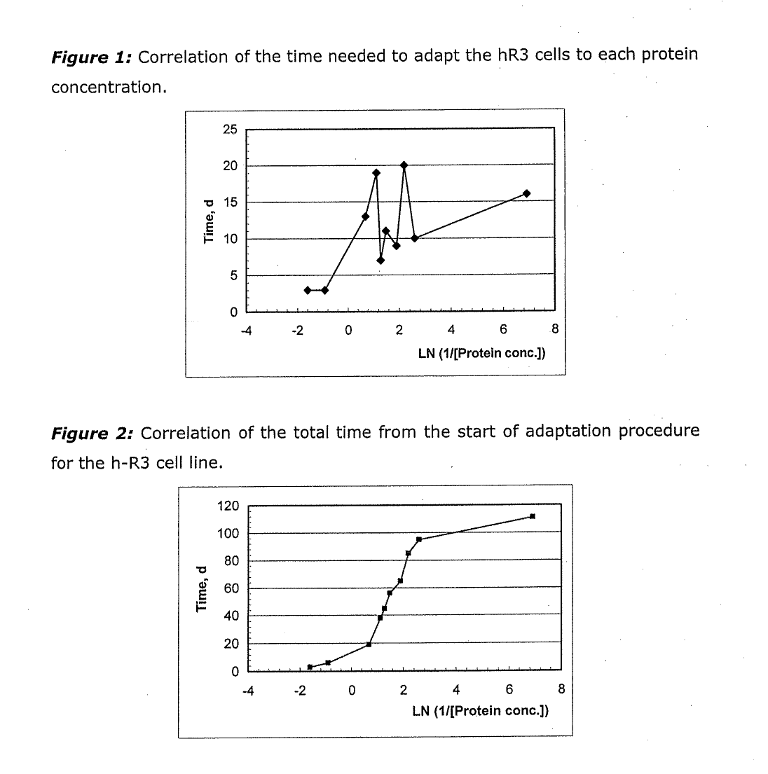

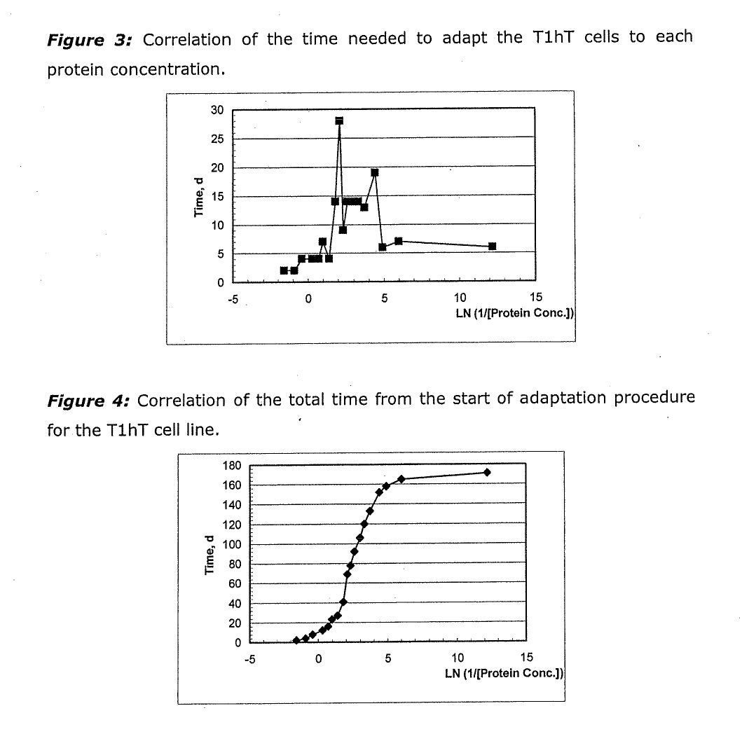

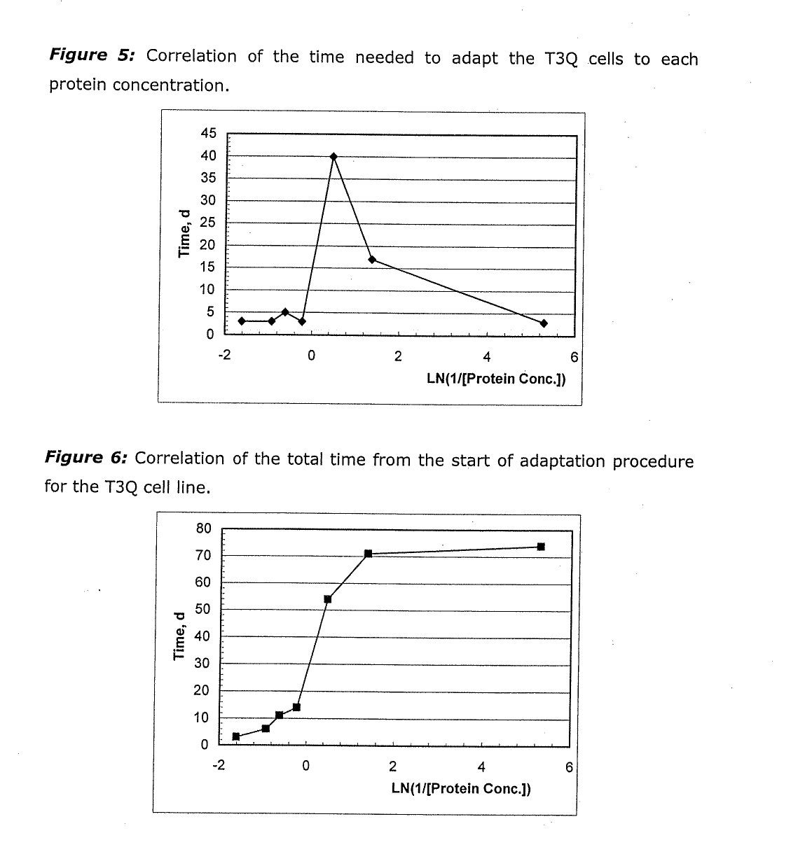

We claim:

1. A method for obtaining a mammalian cell line adapted to growth in a serum and protein free media, which comprises two stages:
I) a first stage wherein the cell line viability is between 80 and 100% and cells are grown in culture media with consecutive protein concentration reduction up to a critical protein concentration at which cell viability drops to 0%; and
II) a second stage wherein once the critical concentration has been predetermined, then a pre-critical concentration is fixed as such protein concentration in which cellular growth is possible and said pre-critical concentration is the start point to slowly reduce the protein concentration up to a point where the cell culture reaches an initial cellular viability and population doubling time.

2. The method according to claim 1 wherein the second stage comprises the following steps:
a) seeding cells from a cell culture with a viability of 80% or higher growing in the pre-critical protein concentration in at least 3 wells at a density in a range of 2 to 6×10⁵ cells/mL;
b) growing the cells in the pre-critical protein concentration and after 48 hours replacing 25% of the supernatant with a fresh supply of the protein-free medium, thus rendering a final protein concentration which is 75% of the pre-critical protein concentration;
c) each 48 hours, completely replacing the supernatant with a fresh culture medium having a protein concentration which is 75% of the pre-critical protein concentration;
d) growing the cells to confluence under said protein concentration;
e) seeding the cells from step (d) in at least 3 wells at a density in the range 2 to 6×10⁵ cells/mL in culture medium with a protein concentration which is 75% of the pre-critical protein concentration;
f) after 48 hours, replacing 25% of the supernatant with a fresh supply of the protein-free medium, thus rendering a final protein concentration which is 75% of the concentration of the previous step;
g) each 48 hours, completely replacing the supernatant with a fresh culture medium with a protein concentration which is 75% of the concentration of the concentration in step (e);
h) growing the cells to confluence under said protein concentration;
i) repeating steps (e) to (h), wherein during each cycle the protein concentration is reduced to 75% of the concentration of the previous cycle, then the procedure being repeated up to the attainment of a protein concentration which does not cause any loss of cell viability and decrease in population doubling time, when the cells are transferred to a medium with lower protein concentration and they are able to grow without any loss of cell viability and decrease in population doubling time before the first subculture, wherein the cells reach the non-critical stage and are seeded directly in the protein-free medium (0 mg/mL of protein concentration).

3. The method according to claim 2, wherein the serum and protein-containing medium in which the cells are initially seeded comprises between 5% and 10% of fetal bovine serum.

4. The method according to claim 3, wherein the mammalian cell line adapted to grow in a serum and protein free media is a myeloma.

5. The method according to claim 4, wherein the myeloma is the NSO cell line.

6. The method according to claim 5, wherein said NSO cell line contains a sequence encoding a recombinant polypeptide or a recombinant protein.

7. The method according to claim 6 wherein the sequence encoding a recombinant polypeptide or a recombinant protein codifies for a recombinant antibody or a fragment thereof.

8. The method according to claim 7 wherein the sequence codifies for the humanized recombinant antibody anti-EGF-R hR3 or a fragment thereof.

9. The method according to claim 7 wherein the sequence codifies for the humanized recombinant anti-CD6 antibody T1hT or a fragment thereof.

10. The method according to claim 7 wherein the sequence codifies for the chimeric recombinant anti-CD3 antibody T3Q or a fragment thereof.
